(19) United States
(12) Patent Application Publication
(10) Pub. No.: US 2019/0351443 A1
(43) Pub. Date: Nov. 21, 2019

(54) VAPORIZER WITH CLOG-FREE CHANNEL

(71) Applicant: INDOSE INC., Woodland Hills, CA (US)

(72) Inventor: Daniel FREEMAN, Woodland Hills, CA (US)

(73) Assignee: INDOSE INC., Woodland Hills, CA (US)

(21) Appl. No.: 16/415,362

(22) Filed: May 17, 2019

Related U.S. Application Data
(60) Provisional application No. 62/672,834, filed on May 17, 2018.

(51) Int. Cl. B05B 15/52; B05B 7/04

(52) U.S. Cl. CPC B05B 15/52; B05B 7/0433

(57) ABSTRACT

A vaporizer includes a housing that comprises a first end and a second end. An atomizer is disposed at the first end of the housing. A mouthpiece is disposed at the second end of the housing. A channel is disposed inside the housing and extends from the first end to the second end. The channel is configured to permit air flow from the atomizer to the mouthpiece. The channel includes a cross section that is substantially non-circular.

### CROSS-REFERENCE TO RELATED APPLICATION

This application claims priority under 35 U.S.C. § 119 to U.S. Application No. 62/672,834 filed on May 17, 2018, in the United States Patent & Trademark Office, the disclosure of which is incorporated herein by reference in its entirety.

### BACKGROUND

Vaporizers are used to vaporize a substance, generally a drug, to give the user the ability to inhale the drug. Generally, the vaporized substance is stored in liquid form in a storage area. The atomizer then changes the state of the liquid into a gas or vapor or aerosol, as to be suspended in air. The user then inhales this substance. One of the advantages of vaporizers is that the substance is quickly delivered to the user's lungs and blood stream. This process is considered healthier that traditional smoking methods that combust the substance.

One issue with vaporizers is that many of them suffer from clogging. This problem is caused by the vapor condensing inside the channel that connects the atomizer to the mouthpiece. The vapor gradually condenses, and deposits on available surfaces and essentially returns to a liquid form in a new location. This usually happens in the above mentioned channel. In such cases that the substance is a viscous liquid, it has the ability to collect and build up. It also gathers to itself due to surface tension. Eventually it can build up in one area so much that it creates a restriction or clog that blocks the flow of air and vapor. This can render the unit useless.

### SUMMARY

According to an aspect of the disclosure, a vaporizer includes a housing that comprises a first end and a second end; an atomizer that is disposed at the first end of the housing; a mouthpiece that is disposed at the second end of the housing; and a channel disposed inside the housing that extends from the first end to the second end, and that is configured to permit air flow from the atomizer to the mouthpiece, wherein the channel includes a cross section that is substantially non-circular, and wherein the channel is configured to cause liquid, formed by the vaporized substance, to flow towards a predetermined area of the channel by capillary action based on an adhesive force between the liquid and a surface of the predetermined area.

### BRIEF DESCRIPTION OF THE DRAWINGS

FIG. 1 is a diagram of a vaporizer with a substantially circular channel;

FIG. 2 is a diagram of a clog progression of a vaporizer with a substantially circular channel;

FIG. 3 is a diagram of a vaporizer with a substantially non-circular channel;

FIG. 4 shows a clog progression of a vaporizer with a substantially non-circular channel; and

FIG. 5 is a diagram of example channel cross sections.

### DETAILED DESCRIPTION

The clogging issue is dependent on various factors such as atomization method, air temperature, substance viscosity, channel temperature, airflow pressure, airflow speed, airflow pathway characteristics, channel material, etc. In fact, it is quite natural that the vapor will want to return to its liquid state, especially as the environment returns to its initial state. One may argue different approaches to avoiding the above mentioned clogging issue. For example, one may try to avoid the condensation altogether in order to avoid the clogging. The present disclosure provides a method for managing the condensation in a way as to avoid a clog.

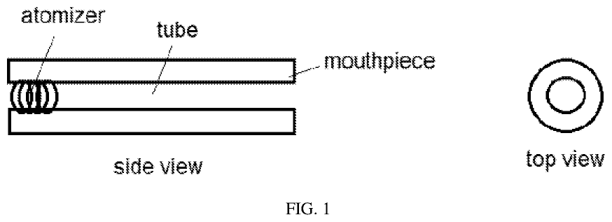

FIG. 1 is a diagram of a vaporizer with a substantially circular channel. As shown in FIG. 1, and by the side view, a vaporizer includes a housing, an atomizer that is disposed at a first end of the housing, a mouthpiece that is disposed at a second end of the housing, and a channel that extends from the housing to the mouthpiece. As shown by the top view, the housing includes a circular cross section. Further, as shown by the top view, the channel includes a circular cross section.

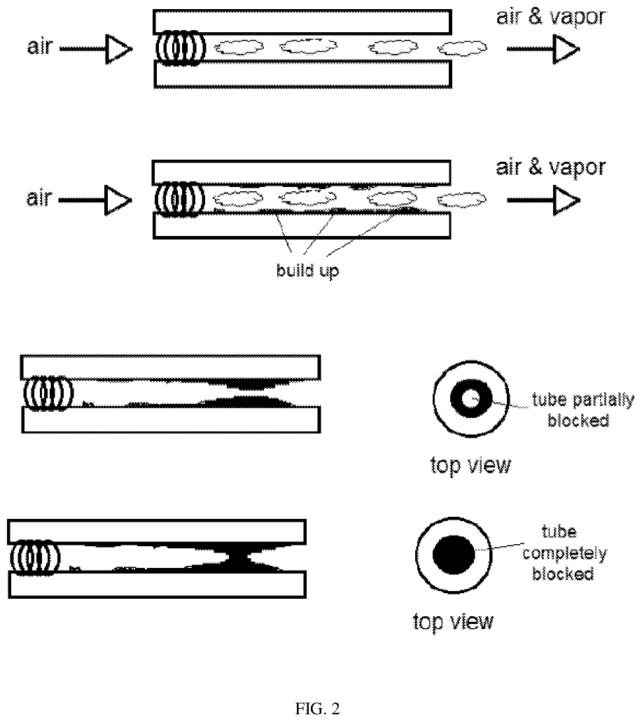

FIG. 2 is a diagram of a clog progression of a vaporizer with a substantially circular channel. During usage of the vaporizer, a user may interact with the mouthpiece to cause air flow from the atomizer to the mouthpiece. In this way, the atomizer may generate vapor, and the user may inhale the vapor by interacting with the mouthpiece to cause air flow from the atomizer to the mouthpiece.

As shown in FIG. 2, as vapor passes through the channel, the vapor re-condenses and aggregates on the circumference of the channel. The manner of aggregation or the degree thereof depends on a variety of forces. Without subscribing to any particular theory, these forces are considered to include, among other things, capillary action (e.g., capillarity, capillary motion, capillary effect, wicking, etc.), adhesive forces between the condensed vapor and the surface of the channel, viscosity of the liquid, and the effect of surface tension. The depositions also depend on general movement, and air flow. Although the invention will be described with reference to the above effects, it will be understood that the invention is not dependent upon whether these effects are scientifically correctly explained herein, as the results described herein are empirically observable in the practice of the invention, without regard to the particular theory of their causation.

Eventually, the condensed vapor aggregates into a localized clog around the circumference of the channel. Further, the condensed vapor finally aggregates into a blockage, thereby entirely clogging the channel.

As shown, the liquid is free to move in both a radial direction and a longitudinal (up and down the channel) direction. Once the liquid moves to clog the channel, the system becomes essentially useless. It may be possible to suck through the clog and clear the channel temporarily. However, the clog will come together once again under the forces of surface tension.

Upon further inspection, it can be seen that that the entire channel does not fill up with oil, but rather only a section of the channel has a clog in it. The liquid spreads somewhat within the channel via capillary action, but eventually clogs.

From a build-up perspective, a circular channel is disadvantageous because any and all deposits are reducing the diameter and creating a smaller distance between opposing sides.

This disclosure addresses the issues described herein by managing the buildup in a way which will avoid a clog. The approach is to allow the condensation to take place and then to control and avoid buildup that will block the channel by providing areas of increased fluid flow, deemed to be due to increased capillarity, e.g., areas where there are narrowings which will provide a greater degree of capillary action, and by the effect of surface tension, pull along small masses of the liquid to distribute it over a greater area than would otherwise be the case in a circular tube.

A vaporizer with a substantially circular cross section is considered to be disadvantageous because the adhesive forces between the channel and the liquid which contribute to capillarity are the same throughout its cross section. This means that the liquid will only travel by capillarity to a certain (and same) extent across the cross section and a buildup will rather easily occur.

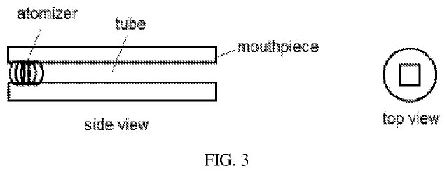

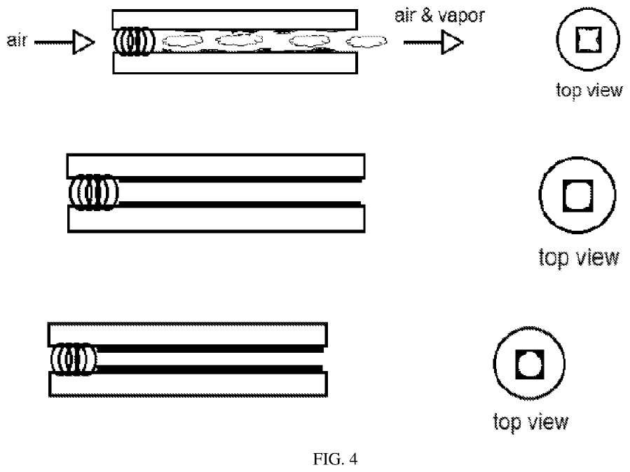

FIG. 3 is a diagram of a vaporizer with a substantially non-circular channel. An embodiment of this disclosure utilizes a channel with a cross section that is substantially non-circular. For example, by using a square cross sectional channel, the build up can be better controlled and managed by the greater capillarity afforded at the narrowings at the corners which will tend to cause the liquid to travel or disperse to a greater extent than is the case with a circular cross section. FIG. 4 shows clog progression of a vaporizer with a substantially non-circular channel. As the vapor passes through the channel, it re-condenses and builds up on the circumference of the channel (much as it does in a round channel). At this point, however, there is a difference with regards to the results of capillary action and adhesive force. The liquid is pulled into the narrowed corners of the square by capillary action and surface tension (e.g., by cohesive force of the molecules of the liquid, and by adhesive force between the liquid and the channel). The corners of a non-cylindrical channel provide a larger surface area for the liquid to adhere to as compared to a cylindrical channel while at the same time creating narrowings which increase capillarity. In this way, capillary action causes the liquid to flow in a corner of the channel, thereby spreading any aggregation over a greater area. Further, surface tension causes some of the liquid to follow the capillary flow. Further still, it is considered that adhesive forces between the liquid and the channel cause the liquid to tend to remain in the corner of the channel.

The channel is configured to cause liquid, formed by condensation of the vaporized substance, to flow into certain areas by capillary action. For example, the vaporized substance condenses to liquid, and flows via capillary action in a predetermined area of the channel such as a corner. In this way, the liquid spreads in the predetermined area via capillary action to a greater extent than in a circular tube, and remains adhered to the predetermined area based on the adhesive force between the liquid and the surface of the predetermined area of the channel.

In the corners, the ratio of: (surface-area)/(volume) is larger than on flat/rounded surfaces, and at the same time there is a narrowing of the surrounding walls, and hence capillary action, surface tension, and adhesive force act to pull the liquid into the corners and to keep the liquid in the corners. It is important to note that this cross sectional representation actually occurs along the entire length of the channel.

As use progresses, the buildup and accumulation will occur in the corners as seen in the top view. It is important to note that the four corners seen in the top view run the entire length of the channel. This represents a significant volume of liquid that is “held” in the corners and is not blocking the flow in the channel.

The volume held in the corners is substantially more than the amount that could create a blockage in a circular channel. It is important to note that after much use, the square channel would accumulate so much liquid that it may start to encroach on the effective diameter of the channel and would restrict flow. It should also be understood that in a disposable vaporizer (a vaporizer designed to be discarded after a set amount of usage) embodiments in this disclosure can trap and distribute a sufficient amount of liquid as to avoid a blockage.

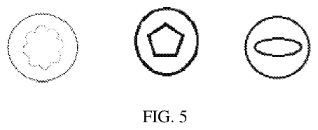

FIG. 5 is a diagram of example channel cross sections. This disclosure is not limited to square channels. Other shapes may include oval, flower, pentagons, triangles and so forth. Shapes that provide areas of a large ratio of (surface-area)/volume and narrowings are especially advantageous. The shapes may be different along the length of the channel, or changing in the radial orientation. The channels may have features throughout their lengths designed to capture and hold the condensed liquid (for example: dimples, steps, lines, waves, protrusions, and so on). Shapes that provide areas of a large ratio of (surface-area)/volume are especially advantageous as noted.

In some implementations, a vaporizer includes a housing that comprises a first end and a second end. An atomizer is disposed at the first end of the housing. A mouthpiece is disposed at the second end of the housing. A channel is disposed inside the housing and extends from the first end to the second end. The channel is configured to permit air flow from the atomizer to the mouthpiece. The channel includes a cross section that is substantially non-circular.

In some implementations, the cross section is substantially non-circular. For example, the cross section may be square, oval, triangular, hexagonal, grooved, polygonal, non-convex polygonal (e.g., a star shape, a flower shape, etc.), and/or the like.

In some implementations, the channel may include a dimple, a step, a line, a groove, a wave, protrusion and/or the like.

In some implementations, the channel includes the cross section along a first length of the channel, and includes another cross section along a second length of the channel, and wherein the first cross section is different than the other cross section.

Even though particular combinations of features are recited in the claims and/or disclosed in the specification, these combinations are not intended to limit the disclosure of possible implementations. In fact, many of these features may be combined in ways not specifically recited in the claims and/or disclosed in the specification. Although each dependent claim listed below may directly depend on only one claim, the disclosure of possible implementations includes each dependent claim in combination with every other claim in the claim set.

No element, act, or instruction used herein should be construed as critical or essential unless explicitly described as such. Also, as used herein, the articles “a” and “an” are intended to include one or more items, and may be used interchangeably with “one or more.” Furthermore, as used herein, the term “set” is intended to include one or more items (e.g., related items, unrelated items, a combination of related and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

What is claimed is:

1. A vaporizer configured to vaporize a substance, comprising:
a housing that comprises a first end and a second end;
an atomizer that is disposed at the first end of the housing;
a mouthpiece that is disposed at the second end of the housing; and
a channel disposed inside the housing that extends from the first end to the second end, and that is configured to permit air flow from the atomizer to the mouthpiece, wherein:
the channel includes a cross section that is substantially non-circular,
the channel is configured to cause liquid, formed by condensation of the vaporized substance, to flow in a predetermined area of the channel to spread to a greater degree than in a circular channel.

2. The vaporizer of claim 1, wherein the cross section is square.

3. The vaporizer of claim 1, wherein the cross section is oval.

4. The vaporizer of claim 1, wherein the cross section is triangular.

5. The vaporizer of claim 1, wherein the cross section is hexagonal.

6. The vaporizer of claim 1, wherein the cross section is polygonal.

7. The vaporizer of claim 1, wherein the cross section is non-convex polygonal.

8. The vaporizer of claim 1, wherein the channel includes at least one of a dimple, a step, a line, a groove, a wave, and a protrusion.

9. The vaporizer of claim 1, wherein the channel includes the cross section along a first length of the channel, and includes another cross section along a second length of the channel, and wherein the cross section is shaped differently than the other cross section.
